SEQUENCE LISTING (20 sequences)

SEQ ID NO: 1 - DNA, 339 bp, Artificial Sequence
  Description of Artificial Sequence Antibody sequence
gacattgtgatgacccaatctccagactctttggctgtgtctctaggggagagggccaccatcaactgcaagtccagtcagagccttttatattctagaaatcaaaagaactacttggcctggtatcagcagaaaccaggacagccacccaaactcctcatcttttgggctagcactagggaatctggggtacctgataggttcagtggcagtgggtttgggacagacttcaccctcaccattagcagcctgcaggctgaagatgtggcagtttattactgtcagcaatattttagctatccgctcacgttcggacaagggaccaaggtggaaataaaa

SEQ ID NO: 2 - protein, 113 aa, Artificial Sequence
  Description of Artificial Sequence Antibody sequence
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 3 - DNA, 339 bp, Artificial Sequence
  Description of Artificial Sequence Antibody sequence
gacattgtgatgacccaatctccagactctttggctgtgtctctaggggagagggccaccatcaactgcaagtccagtcagagccttttatattctagaaatcaaaagaactacttggcctggttccagcagaaaccaggacagccacccaaactcctcatcttttgggctagcactagggaatctggggtacctgataggttcagtggcagtgggtttgggacagacttcaccctcaccattagcagcctgcaggctgaagatgtggcagtttatgactgtcaacaatattttagctatccgctcacgttcggacaagggaccaaggtggaaataaaa

SEQ ID NO: 4 - protein, 113 aa, Artificial Sequence
  Description of Artificial Sequence Antibody sequence
DIVMTQSPDSLAVSLGERATINCKSSQSLLYSRNQKNYLAWFQQKPGQPPKLLIFWASTRESGVPDRFSGSGFGTDFTLTISSLQAEDVAVYDCQQYFSYPLTFGQGTKVEIK

SEQ ID NO: 5 - DNA, 339 bp, Artificial Sequence
  Description of Artificial Sequence Antibody sequence
gacattgtgatgacccaatctccagactctttggctgtgtctctaggggagagggccaccatcaactgcaagtccagtcagagccttttatattctagaaatcaaaagaactacttggcctggtatcagcagaaaccaggacagccacccaaactcctcatctattgggctagcactagggaatctggggtacctgataggttcagtggcagtgggtttgggacagacttcaccctcaccattagcagcctgcaggctgaagatgtggcagtttattactgtcagcaatattttagctatccgctcacgttcggacaagggaccaaggtggaaataaaa

SEQ ID NO: 6 - protein, 113 aa, Artificial Sequence
  Description of Artificial Sequence Antibody sequence
DIVMTQSPDSLAVSLGERATINCKSSQSLLYSRNQKNYLAWYQQKPGQPPKLLIYWASTRESGVPDRFSGSGFGTDFTLTISSLQAEDVAVYYCQQYFSYPLTFGQGTKVEIK

SEQ ID NO: 7 - DNA, 372 bp, Artificial Sequence
  Description of Artificial Sequence Antibody sequence
caggtgcaactagtgcagtccggcgccgaagtgaagaaacccggtgcttccgtgaaagtcagctgtaaaactagtagatacaccttcactgaatacaccatacactgggttagacaggcccctggccaaaggctggagtggataggaggtattaatcctaacaatggtattcctaactacaaccagaagttcaagggccgggccaccttgaccgtaggcaagtctgccagcaccgcctacatggaactgtccagcctgcgctccgaggacactgcagtctactactgcgccagaagaagaatcgcctatggttacgacgagggccatgctatggactactggggtcaaggaacccttgtcaccgtctcctca

SEQ ID NO: 8 - protein, 124 aa, Artificial Sequence
  Description of Artificial Sequence Antibody sequence
QVQLVQSGAEVKKPGASVKVSCKTSRYTFTEYTIHWVRQAPGQRLEWIGGINPNNGIPNYNQKFKGRATLTVGKSASTAYMELSSLRSEDTAVYYCARRRIAYGYDEGHAMDYWGQGTLVTVSS

SEQ ID NO: 9 - DNA, 372 bp, Artificial Sequence
  Description of Artificial Sequence Antibody sequence
caggtgcaactagtgcagtccggcgccgaagtgaagaaacccggtgcttccgtgaaagtcagctgtaaaactagtagatacaccttcactgaatacaccatacactgggttagacaggcccctggccaaaggctggagtggataggaggtattaatcctaacaatggtattcctaactacaaccagaagttcaagggccgggccaccttgaccgtaggcaagtctgccagcaccgcctacatggaactgtccagcctgcgctccgaggacactgcagtctacttctgcgccagaagaagaatcgcctatggttacgacgagggccatgctatggactactggggtcaaggaacccttgtcaccgtctcctca

SEQ ID NO: 10 - protein, 124 aa, Artificial Sequence
  Description of Artificial Sequence Antibody sequence
QVQLVQSGAEVKKPGASVKVSCKTSRYTFTEYTIHWVRQAPGQRLEWIGGINPNNGIPNYNQKFKGRATLTVGKSASTAYMELSSLRSEDTAVYFCARRRIAYGYDEGHAMDYWGQGTLVTVSS

SEQ ID NO: 11 - DNA, 372 bp, Artificial Sequence
  Description of Artificial Sequence Antibody sequence
caggtgcaactagtgcagtccggcgccgaagtgaagaaacccggtgcttccgtgaaagtcagctgtaaaactagtagatacaccttcactgaatacaccatacactgggttagacaggcccctggccaaaggctggagtggataggaggtattaatcctaacaatggtattcctaactacaaccagaagttcaagggccgggtcaccatcaccgtagacacctctgccagcaccgcctacatggaactgtccagcctgcgctccgaggacactgcagtctactactgcgccagaagaagaatcgcctatggttacgacgagggccatgctatggactactggggtcaaggaacccttgtcaccgtctcctca

SEQ ID NO: 12 - protein, 124 aa, Artificial Sequence
  Description of Artificial Sequence Antibody sequence
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 13 - DNA, 372 bp, Artificial Sequence
  Description of Artificial Sequence Antibody sequence
caggtgcaactagtgcagtccggcgccgaagtgaagaaacccggtgcttccgtgaaagtcagctgtaaaactagtagatacaccttcactgaatacaccatacactgggttagacaggcccctggccaaaggctggagtggataggaggtattaatcctaacaatggtattcctaactacaaccagaagttcaagggccgggtcaccatcaccgtagacacctctgccagcaccgcctacatggaactgtccagcctgcgctccgaggacactgcagtctacttctgcgccagaagaagaatcgcctatggttacgacgagggccatgctatggactactggggtcaaggaacccttgtcaccgtctcctca

SEQ ID NO: 14 - protein, 124 aa, Artificial Sequence
  Description of Artificial Sequence Antibody sequence
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYFCARRRIAYGYDEGHAMDYWGQGTLVTVSS

SEQ ID NO: 15 - DNA, 372 bp, Artificial Sequence
  Description of Artificial Sequence Antibody sequence
caggtgcaactagtgcagtccggcgccgaagtgaagaaacccggtgcttccgtgaaagtcagctgtaaaactagtggatacaccttcactgaatacaccatacactgggttagacaggcccctggccaaaggctggagtggataggaggtattaatcctaacaatggtattcctaactacaaccagaagttcaagggccgggtcaccatcaccgtagacacctctgccagcaccgcctacatggaactgtccagcctgcgctccgaggacactgcagtctactactgcgccagaagaagaatcgcctatggttacgacgagggccatgctatggactactggggtcaaggaacccttgtcaccgtctcctca

SEQ ID NO: 16 - protein, 124 aa, Artificial Sequence
  Description of Artificial Sequence Antibody sequence
QVQLVQSGAEVKKPGASVKVSCKTSGYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 17 - DNA, 321 bp, Artificial Sequence
  Description of Artificial Sequence Antibody sequence
cgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 18 - protein, 107 aa, Artificial Sequence [sequence omitted]

SEQ ID NO: 19 - DNA, 990 bp, Artificial Sequence
  Description of Artificial Sequence Antibody sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 330 aa, Artificial Sequence
  Description of Artificial Sequence Antibody sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2003/0103968 A1
(43) Pub. Date: Jun. 5, 2003

(54) Use of alpha specific antibody BIBH1 in the treatment of cancer

(76) Inventors: Andree Amelsberg, Danbury, CT (US); Andrew Scott, Kew (AU); Paul Tanswell, Laupheim (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/121,464

(22) Filed: Apr. 11, 2002

Related U.S. Application Data
(60) Provisional application No. 60/283,868, filed on Apr. 12, 2001.

(51) Int. Cl. A61K 39/395; C07K 16/40

(52) U.S. Cl. 424/138.1; 424/146.1; 424/155.1; 530/388.26

(57) ABSTRACT

A method of treating a patient suffering from a pathological condition characterized by expression of FAPα, the method comprising administering to the patient a therapeutically effective amount of an antibody which specifically binds to FAPα. A pharmaceutical composition comprising an antibody which specifically binds to FAPα, wherein the antibody is radiolabeled with ¹³¹I, ⁹⁰Y, ¹⁸⁶Re, or ¹⁸⁸Re, and wherein the antibody has specific activity of from about 0.5 to about 15 mCi/mg, is also provided.

### RELATED APPLICATIONS

Benefit under 35 U.S.C. §119(e) of prior U.S. provisional application Serial No. 60/283,868, filed Apr. 12, 2001, is hereby claimed and is hereby incorporated by reference in its entirety.

### REFERENCE TO SEQUENCE LISTING

In accordance with 37 C.F.R. §§1.821-1.824, the application includes a Sequence Listing provided in both paper form and computer readable form (CRF). Both the paper form of the Sequence Listing and computer readable form of the Sequence Listing are hereby incorporated by reference in their entireties herein. The Sequence Listing information recorded in the computer readable form of the Sequence Listing is the same as the Sequence Listing information printed in the paper form of the Sequence Listing.

### FIELD OF THE INVENTION

This invention relates to the use of humanized antibodies which, when labeled with a therapeutic label, is effective as a therapeutic agent. More specifically, the antibodies bind to a molecule known as “fibroblast activation protein alpha” (“FAPα”).

### BACKGROUND OF THE INVENTION

The invasive growth of epithelial cancers is associated with a number of characteristic cellular and molecular changes in the supporting stroma. A highly consistent molecular trait of the reactive stroma of many types of epithelial cancer is induction of the FAPα, a cell surface molecule of reactive stromal fibroblasts that reacts with monoclonal antibody F19 (Garin-Chesa P, et al., Proc. Natl. Acad. Sci. 87: 7235 (1990)). Since the FAPα antigen is selectively expressed in the stroma of a range of epithelial carcinomas, independent of location and histological type, an FAPα-targeting concept has been developed for imaging, diagnosis and treatment of epithelial cancers and certain other conditions. A monoclonal antibody termed F19 that specifically binds to FAPα is described in U.S. Pat. No. 5,059,523 and WO 93/05804, which are hereby incorporated by reference in their entirety.

In immunotherapy of cancer, an antibody is intended to specifically deliver a cytotoxic agent to the tumor, or is toxic itself to the target. After systemic administration, the antibody circulates through the body and accumulates at the tumor site(s). It is important, however, that the antibody fraction not bound to the tumor after the accumulation phase be cleared from the circulation rapidly, in order to minimize unwanted side effects. This is a particular requirement in radioimmunotherapy (RIT) of tumors. On the other hand, if the antibody is cleared before it is effectively taken up by the target site, it will not be sufficiently effective. Half life of the antibody in the circulation depends on the actual dose administered. Furthermore, the actual dose which might be administered to a patient is limited by the maximum tolerated dose, which puts a further constraint on the administration scheme. Thus, the amount of antibody or antibody conjugate which is to be applied for safe and effective treatment depends on a number of parameters which cannot be easily anticipated.

An additional serious problem that arises when using non-human antibodies (i.e. mainly rodent) for applications in vivo in humans is that they quickly raise a human anti-non-human (i.e. anti-rodent) response that reduces the efficacy of the antibody in patients and impairs continued administration. Humanization of non-human antibodies is commonly achieved in one of two ways: (1) by constructing non-human/human chimeric antibodies, wherein the non-human variable regions are joined to human constant regions (Boulianne, et al., Nature 312: 643 (1984)) or (2) by grafting the complementarity determining regions (CDRs) from the non-human variable regions to human variable regions and then joining these “reshaped human” variable regions to human constant regions (Riechmann L, et al, Nature 332: 323 (1988)). Chimeric antibodies, although significantly better than mouse antibodies, can still elicit an anti-non-human (anti-rodent) response in humans (LoBuglio, et al., Proc. Natl. Acad. Sci. 86: 4220 (1984)). CDR-grafted or reshaped human antibodies contain little or no protein sequences that can be identified as being derived from mouse antibodies. Although an antibody humanized by CDR-grafting may still be able to elicit some immune reactions (human-anti-human), such as an anti-allotype or an anti-idiotypic response, the CDR-grafted antibody will be significantly less immunogenic than a non-human (rodent) antibody thus enabling a more prolonged treatment of patients.

Another serious limitation relating to the commercial use of antibodies for diagnosis, imaging, and therapy is their producibility in large amounts. In many instances recombinant expression of native (non-human, i.e. rodent), chimeric and/or CDR-grafted antibodies in cell culture systems is poor. Factors contributing to poor producibility may include the choice of leader sequences and the choice of host cells for production as well as improper folding and reduced secretion. Improper folding can lead to poor assembly of heavy and light chains or a transport incompetent conformation that forbids secretion of one or both chains. It is generally accepted that the L-chain confers the ability of secretion of the assembled protein. In some instances multiple or even single substitutions can result in the increased producibility of antibodies.

Targeting tumor stroma has been recognized as a potential avenue for treatment of cancers. It has been observed that many epithelial cancers, including breast and colon cancer, contain significant amounts of stroma.

There are several issues which confront the artisan who attempts to develop immuno-therapeutic approaches to treatment of conditions like cancer. One of these are the so-called “HAMA” or “HAHA” responses. “HAMA” is an abbreviation for “human anti-mouse antibodies,” where “HAHA” stands for “human anti-human antibodies.” Each of these is a recognized phenomenon which occurs when a human subject receives a dose of murine or human antibodies. The patient's immune system recognizes and attacks the therapeutic agent. The consequences of this can range from inactivation of the agent, to creation of a life threatening situation.

A second issue relates to the use of conjugates of antibodies and therapeutic agents. Various anti-cancer drugs have been attached to antibodies, with the resulting conjugates being administered to the patient. Theoretically, the antibody targets the site of the pathology, and the therapeutic agent is thus directed to a site when it is effective.

A problem with such conjugates is that if they are retained in the patient for extended periods of time, they can damage healthy tissue which they contact. A key example of this is the effect that radioactive labels have in bone marrow cells. The radiolabels destroy the bone marrow cells, leading to thrombocytopenia and anemia as well as leukopenia and neutropenia resulting in immuno-suppression, all with potentially grave consequences.

It is hoped that the humanization reduces the immunogenicity and the risk of immune response-induced rapid clearance of the antibody from the circulation, in particular upon repeated dosing. That this goal is not a trivial one, became evident in a recent clinical trial with the humanized version of the A33 antibody, which was immunogenic in several of the 11 treated patients with advanced colorectal cancer (Welt S and Ritter G, Antibodies in the Therapy of Colon Cancer, Semin Oncol 1999; 26(6):683-690; Ritter G, Cohen LS, Williams C Jr, Richards E C, Old L J, and Welt S, Cancer Res 2001; 61:6851-9).

Hence, the potential usefulness of immunotherapeutic materials must be balanced against the issues that such therapies raise.

How these and other aspects of the invention are achieved will be seen in the disclosure which follows.

### BRIEF DESCRIPTION OF THE DRAWING

FIG. 1: Example serum concentration time-profile of sibrotuzumab (BIBH1) in one patient showing measured data points and 2-compartment model fit.

### DETAILED DESCRIPTION OF THE PREFERRED EMBODIMENTS

Definitions of Important Terms as Used in the Description

Before the embodiments of the present invention it must be noted that as used herein and in the appended claims, the singular forms “a”, “an”, and “the” include plural reference unless the context clearly dictates otherwise. Thus, for example, reference to “an antibody” includes a plurality of such antibodies, reference to the “radioisotope” is a reference to one or more radioisotopes and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

The terms “antibody molecule” or “antibody protein” or “antibody” as used herein shall be considered equivalent.

“Complementarity determining regions of a monoclonal antibody” are understood to be those amino acid sequences involved in specific antigen binding according to Kabat (Kabat et al., 1991) in connection with Chothia and Lesk (Chothia and Lesk (1987).

As used herein, the term “framework modifications” refers to the exchange, deletion or addition of single or multiple amino acids in the variable regions surrounding the individual complementarity determining regions. Framework modifications may have an impact on the immunogenicity, producibility or binding specificity of an antibody protein.

A “fragment” according to the invention is a shorter antibody molecule, i.e. any polypeptide subset, characterized in that it is encoded by a shorter nucleic acid molecule than disclosed below, however still retains its antibody binding activity.

A “functional variant” of the antibody molecule according to the invention is a antibody molecule which possesses a biological activity (either functional or structural) that is substantially similar to the antibody molecule according to the invention, i.e. a substantially similar substrate specificity or cleavage of the substrate. The term “functional variant” also includes “a fragment”, “an allelic variant”, “a functional variant”, “variant based on the degenerative nucleic acid code” or “chemical derivatives”. Such a “functional variant” e.g. may carry one or several point mutations, one or several nucleic acid exchanges, deletions or insertions or one or several amino acid exchanges, deletions or insertions. Said functional variant still retains its biological activity, such as antibody binding activity, at least in part, or even going along with an improvement said biological activity.

A “functional variant” of the antibody molecule according to the invention is a antibody molecule which possesses a biological activity (either functional or structural) that is substantially similar to the antibody molecule according to the invention, i.e. a substantially similar target molecule binding activity. The term “functional variant” also includes “a fragment”, “an allelic variant”, “a functional variant”, “variant based on the degenerative nucleic acid code” or “chemical derivatives”.

An “allelic variant” is a variant due to the allelic variation, e.g. differences in the two alleles in humans. Said variant is still retaining its biological activity such as antibody target binding activity, at least in part or even going along with an improvement said biological activity.

A “variant based on the degenerative of the genetic code” is a variant due to the fact that a certain amino acid may be encoded by several different nucleotide triplets. Said variant is still retaining its biological activity such as antibody binding activity, at least in part or even going along with an improvement said biological activity.

A “fusion molecule” may be the antibody molecule according to the invention fused to e.g. a reporter such as a radiolabel, a chemical molecule such as a toxin or a fluorescent label or any other molecule known in the art.

As used herein, a “chemical derivative” according to the invention is a antibody molecule according to the invention chemically modified or containing additional chemical moieties not normally being part of the molecule. Such moieties may improve the molecule's activity such as target destruction (e.g. killing of tumor cells) or may improve its solubility, absorption, biological half life etc.

A molecule is “substantially similar” to another molecule if both molecules have substantially similar structures or biological activity. Thus, provided that two molecules possess a similar activity, they are considered variants as that term is used herein even if the structure of one of the molecules is not found in the other, or if the sequence of amino acid residues is not identical.

For many uses of the antibodies according to the invention it is desirable to have the smallest possible antigen-binding, i.e. FAPα-binding units. Therefore, in another preferred embodiment, an antibody protein according to the invention is a Fab fragment (Fragment antigen-binding=Fab). These FAPα-specific antibody proteins according to the invention consist of the variable regions of both chains which are held together by the adjacent constant region. These may be formed by protease digestion, e.g. with papain, from conventional antibodies, but similar Fab fragments may also be produced in the mean time by genetic engineering.

Using genetic engineering methods it is possible to produce shortened antibody fragments which consist only of the variable regions of the heavy (VH) and of the light chain (VL). These are referred to as Fv fragments (Fragment variable=fragment of the variable part). In another preferred embodiment an FAPα-specific antibody molecule according to the invention is such an Fv fragment. Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins of this kind known from the prior art are described in Huston et al (1988).

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerization of the scFv, scFv were prepared as fusion proteins with multimerization domains. The multimerization domains may be, e.g. the CH3 region of an IgG or coiled coil structure (helix structures) such as Leucin-zipper domains. However, there are also strategies in which the interaction between the VH/VL regions of the scfv are used for the multimerization (e.g. di-, tri-, and pentabodies).

By “diabody” the skilled person means a bivalent homodimeric scFv derivative (Huston et al., 1996; Perisic et al. 1994; Hu et al. 1996). The shortening of the Linker in an scFv molecule to 5-10 amino acids leads to the formation of homodimers in which an inter-chain VH/VL-superimposition takes place. Diabodies may additionally be stabilized by the incorporation of disulfide bridges. Examples of diabody-antibody proteins from the prior art can be found in Perisic (1994).

By “minibody” the skilled person means a bivalent, homodimeric scFv derivative. It consists of a fusion protein which contains the CH3 region of an immunoglobulin, preferably IgG, most preferably IgGI as the dimerization region which is connected to the scFv via a Hinge region (e.g. also from IgGI) and a Linker region. The disulfide bridges in the Hinge region are mostly formed in higher cells and not in prokaryotes. In another preferred embodiment an antibody protein according to the invention is an FAPα-specific minibody antibody fragment. Examples of minibody-antibody proteins from the prior art can be found in Hu et al. (1996).

By “triabody” the skilled person means a trivalent homotrimeric scFv derivative (Kortt et al. 1997). ScFv derivatives wherein VH-VL are fused directly without a linker sequence lead to the formation of trimers.

The skilled person will also be familiar with so-called miniantibodies which have a bi-, tri- or tetravalent structure and are derived from scFv. The multimerization is carried out by di-, tri- or tetrameric coiled coil structures (Pack et al., 1993; Lovejoy et al. 1993; Pack et al., 1995).

“Antibody protein” according to the invention includes Fab-or F(ab′)₂-fragments of immunoglobulins, single-chain-antibodies (scFv), chimeric or humanized antibodies or entirely human antibodies produced by e.g. recombinant methods. “Antibody protein” according to the invention also relates to fragments, allelic variants, functional variants, glycosylation variants, fusion molecules or a chemical derivatives thereof. Definitions of such derivatives may be found supra.

Disclosure of the Embodiments of the Invention

In one embodiment the present invention relates to the use of antibody proteins having the complementary determining regions of the monoclonal antibody F19 (ATCC Accession No. HB 8269), said new antibody proteins specifically binding to fibroblast activation protein a (FAPα), characterized in that they have framework modifications resulting in the improved producibility in host cells as compared to a chimeric antibody having the variable regions of F19 and foreign constant regions, wherein said antibody protein is derived from the murine antibody designated F 19 (ATCC Accession No. HB 8269).

To generate humanized FAP-specific antibody proteins a chimeric antibody was constructed, having variable regions of the light and heavy chains of F 19 and human light and heavy constant regions, respectively. The construction and production of chimeric mouse/human antibodies is well known (Boulianne et al. (1984), referenced above).

The variable regions of the antibody proteins of the present invention are typically linked to at least a portion of the immunoglobulin constant region (Fc), typically that of a human immunoglobulin. Human constant region DNA sequences can be isolated in accordance with well-known procedures from a variety of human cells, but preferably immortalized B cells (see Kabat et al., supra, and WO 87/02671). Hence the antibody proteins of the invention may contain all or only a portion of the constant region as long as they exhibit specific binding to the FAP antigen. The choice of the type and extent of the constant region depends on whether effector functions like complement fixation or antibody dependent cellular toxicity are desired, and on the desired pharmacological properties of the antibody protein. The antibody protein of the invention will typically be a tetramer consisting of two light chain/heavy chain pairs, but may also be dimeric, i.e. consisting of a light chain/heavy chain pair, e.g. a Fab or Fv fragment.

Therefore, in a further embodiment the invention relates to the use of antibody proteins according to the invention, characterized in that they have a variable light chain region and a variable heavy chain region, each joined to a human constant region. In particular, the variable region of the light chain was joined to a human kappa constant region and the variable region of the heavy chain was joined to a human gamma-1 constant region. Other human constant regions for humanizing light and heavy chains are also available to the expert.

Therefore, in one particular embodiment the antibody proteins, which are used according to the invention, contain a human kappa constant region.

Also, in another particular embodiment the antibody proteins, which are used according to the invention, contain a human gamma-1 constant region.

While the mutation of the leader sequence only leads to a doubling of the expression yield of the chimeric F19 antibody, a substantial improvement as defined herein refers to an improvement in expression level and/or purification yield of at least a factor of 10.

In a preferred embodiment, the invention refers to antibody proteins, to be used according to the invention, are characterized in that their expression levels in crude media samples as determined by ELISA and/or purified antibody yields exceed the expression levels and/or purification yields of the chimeric antibodies without framework modifications by at least a factor of 10.

In a more preferred embodiment, the invention refers to antibody proteins, to be used according to the invention, wherein said antibody proteins are characterized in that their expression levels in crude media samples as determined by ELISA and/or purified antibody yields exceed the expression levels and/or purification yields of the chimeric antibodies without framework modifications by at least a factor of 20.

In a most preferred embodiment, antibody proteins, to be used according to the invention, are characterized in that their expression levels in crude media samples as determined by ELISA and/or purified antibody yields exceed the expression levels and/or purification yields of the chimeric antibodies without framework modifications by at least a factor of 100.

Improved producibility of the recombinant antibody proteins of the invention can be demonstrated for eukaryotic cells in general as shown for COS and CHO (Chinese hamster ovary derived cells) eukaryotic cells. In a further embodiment, the present invention relates to the use of recombinant antibody proteins characterized in that they display improved producibility in eukaryotic cells.

In a preferred embodiment the present invention relates to antibody proteins, wherein said eukaryotic cell is a Chinese hamster ovary cell (CHO cell).

It was unexpectedly found that certain framework modifications of the light chain variable regions determine the improved producibility of the antibody proteins of the invention. Three versions of reshaped light chain variable regions, designated version A, B, and C, as described below, were prepared.

Any of the following sequences may be comprised in an antibody according to the invention. Said antibodies are specifically binding to FAPα.

The DNA sequence of F19 human reshaped light chain variable region version A (hIF19L_{A}) is disclosed in SEQ ID NO: 1. The amino acid sequence of F19 human reshaped light chain variable region version A (hF19L_{A}) is disclosed in SEQ ID NO: 2.

The DNA sequence of F 19 human reshaped light chain variable region version B (hF19L_{B}) is disclosed in SEQ ID NO: 3. The amino acid sequence of F19 human reshaped light chain variable region version B (hF19L_{B}) is disclosed in SEQ ID NO: 4.

The DNA sequence of F 19 human reshaped light chain variable region version C (hF19L_{C}) is disclosed in SEQ ID NO: 5. The amino acid sequence of F19 human reshaped light chain variable region version C (hF19L_{C}) is disclosed in SEQ ID NO: 6.

The DNA sequence of F19 human reshaped variable region heavy chain version A (hF19H_{A}) is disclosed in SEQ ID NO: 7. The amino acid sequence of F19 human reshaped heavy chain variable region version A (hF19H_{A}) is disclosed in SEQ ID NO: 8.

The DNA sequence of F19 human reshaped heavy chain variable region version B (hF19H_{B}) SEQ ID NO: 9. The amino acid sequence of F19 human reshaped heavy chain variable region version B (hF19H_{B}) is disclosed in SEQ ID NO: 10.

The DNA sequence of F19 human reshaped heavy chain variable region version C (hF19H_{C}) SEQ ID NO: 11. The amino acid sequence of F19 human reshaped heavy chain variable region version C (hF19H_{C}) is disclosed in SEQ ID NO: 12.

The DNA sequence of F19 human reshaped heavy chain variable region version D (hF19H_{D}) is disclosed in SEQ ID NO: 13. The amino acid sequence of F19 human reshaped heavy chain variable region version D (hF19H_{D}) is disclosed in SEQ ID NO: 14.

The DNA sequence of F19 human reshaped heavy chain variable region version E (hF19H_{E}) is disclosed in SEQ ID NO: 15. The amino acid sequence of F19 human reshaped heavy chain variable region version E (hF19H_{E}) is disclosed in SEQ ID NO: 16.

The DNA sequence of human kappa light constant chain is disclosed in SEQ ID NO: 17.

The amino acid sequence of human light constant chain is disclosed in SEQ ID NO: 18.

The DNA sequence of human heavy constant chain is disclosed in SEQ ID NO: 19.

The amino acid sequence of human heavy constant chain is disclosed in SEQ ID NO: 20.

Light chain variable region versions A, B, and C demonstrate substantially improved producibility in CHO cells. While light chain variable region versions A and C differ from light chain variable region version B by only two common amino acid residues they display an even further substantial improvement in producibility. There is at least another 10 fold difference in antibody secretion levels between the human reshaped F19 light chain version B and versions A or C. Reshaped human F19 light chain version A and B only differ in their amino acid sequences by two residues at positions 36 (Tyr to Phe mutation) and 87 (Tyr to Asp mutation) (nomenclature according to Kabat). This negative effect on the secretory capability of antibodies containing the light chain variable region version B could have been indirect if the Tyr to Asp and Tyr to Phe mutations, considered individually or together, merely caused improper folding of the protein. But this is unlikely to be the case since antigen binding assays show that immunoglobulins containing F19 light chain version B have similar avidities to those paired with F19 light chain version A or C, suggesting that they were not grossly misfolded.

Residue 87 in reshaped human F19 light chain version B seems particularly responsible for the reduction of secretion when compared to versions A and C.

In a preferred embodiment, the present invention relates to the use of antibody proteins according to the invention, wherein the amino acid in Kabat position 87 of the light chain region is not asparagine.

In a more preferred embodiment, the invention relates to the use of antibody proteins according to the invention, wherein the amino acid in Kabat position 87 of the light chain region is selected from aromatic or aliphatic amino acids.

In a most preferred embodiment, the present invention relates to the use of antibody proteins according to the invention, wherein the aromatic amino acid in Kabat position 87 of the light chain region is a tyrosine or phenylalanine.

In a further embodiment, the present invention also pertains to the use of antibody proteins according to the invention, wherein the amino acid in Kabat position 36 of the light chain region is selected from aromatic amino acids.

In a particular embodiment the invention relates to the use of the specific antibody proteins that may be prepared from the individually disclosed reshaped variable regions of the light and heavy chains.

Especially light chain variable region versions A and C are particularly suitable to practice the invention because of their exceptionally high producibility, while retaining full FAP-binding specificity and achieving low immunogenicity. This holds especially true when compared to the chimeric antibody having the variable regions of F19 and the same constant regions but also when compared to light chain version B.

Therefore, in one embodiment the present invention relates to the use of antibody proteins that contain the variable region of the light chain selected from the group as set forth in SEQ ID NO: 2, SEQ ID NO: 4 or SEQ ID NO: 6.

In a preferred embodiment the present invention relates to the use of antibody proteins that contain the variable region of the light chain as set forth in SEQ ID NO: 2.

In a further embodiment the invention also relates to the use of antibody proteins, characterized in that the variable region of the light chain is encoded by a nucleotide sequence selected from the group as set forth in SEQ ID NO: 1, SEQ ID NO: 3 or SEQ ID NO: 5.

In a further embodiment the invention also relates to the use of antibody proteins, characterized in that the variable region of the light chain selected from the group is encoded by a nucleotide sequence as set forth in SEQ ID NO: 1.

In a preferred embodiment the present invention relates to the use of antibody proteins that contain the variable region of the light chain selected from the group as set forth in SEQ ID NO: 6.

In a further preferred embodiment the invention also relates to antibody proteins characterized in that the variable region of the light chain is encoded by a nucleotide sequence as set forth in SEQ ID NO: 5.

The present invention also discloses several different variable regions of the heavy chain that work particularly well with the variable regions of the light chain versions A and C in terms of improved producibility.

In one embodiment the invention relates to the use of antibody proteins containing a variable region of the heavy chain as set forth in any one of SEQ ID NOs: 8, 10, 12, 14, 16.

In another embodiment the invention relates to the use of antibody proteins characterized in that the variable region of the heavy chain is encoded by a nucleotide sequence as set forth in any one of SEQ ID NOs: 7, 9, 11, 13, 15.

In one embodiment the invention relates to the use of antibody proteins containing a constant region of the light chain as set forth in SEQ ID NO: 18.

In one embodiment the invention relates to the use of antibody proteins containing a constant region of the heavy chain as set forth in SEQ ID NO: 20.

In another embodiment the invention relates to the use of antibody proteins characterized in that the constant region of the light chain is encoded by a nucleotide sequence as set forth in SEQ ID NO: 17.

In another embodiment the invention relates to the use of antibody proteins characterized in that the constant region of the heavy chain is encoded by a nucleotide sequence as set forth in SEQ ID NO: 19.

In a very particular embodiment the invention relates to the use of antibody proteins containing the variable region of the light chain as set forth in SEQ ID NO: 2 and the variable region of the heavy chain as set forth in SEQ ID NO: 12. Most preferably, this antibody protein additionally contains the constant region of the light chain as set forth in SEQ ID NO: 18 and the constant region of the heavy chain as set forth in SEQ ID NO: 20.

The humanized antibody described herein, and used in the invention, i.e. BIBH 1 (INN: sibrotuzumab), has been described in PCT application WO 99/57151, the disclosure of which is incorporated by reference in its entirety. SEQ ID NO: 1 of this application sets forth the light chain variable region of BIBH 1. Thus a further aspect of the present invention is the use of an antibody protein containing an amino acid sequence as set forth in SEQ ID NO: 2. More preferably, such an antibody protein further contains an amino acid sequence as set forth in SEQ ID NO: 12. More preferably, said antibody protein further contains an amino acid sequence as set forth in SEQ ID NO: 20 and an amino acid sequence as set forth in SEQ ID NO: 22. These make up the BIBH 1 antibody, and are set forth in this application. The target of this antibody, known in the art as FAPα, is disclosed in, e.g., U.S. Pat. Nos. 5,965,373; 5,767,242; and 5,587,299, all of which are incorporated by reference.

Humanization of the variable region of a murine antibody may be achieved employing methods known in the art. EP 0239400 discloses grafting of the CDRs of a murine variable region into the framework of a human variable region. WO 90/07861 discloses methods of reshaping a CDR-grafted variable region by introducing additional framework modifications. WO 92/11018 discloses methods of producing humanized Ig combining donor CDRs with an acceptor framework that has a high homology to the donor framework. WO 92/05274 discloses the preparation of framework mutated antibodies starting from a murine antibody. Further prior art references related to humanization of murine monoclonal antibodies are EP 0368684; EP 0438310; WO 92/07075, or WO 92/22653. Thus, the expert can produce the antibodies of the present invention starting from the publicly available murine monoclonal antibody F19 and employing techniques known in the art, e.g. from WO 92/05274. DNA molecules coding for the antibody proteins of the present invention may of course also be obtained by state-of-the-art synthetic procedures, e.g. by chemical synthesis of appropriate oligonucleotides and subsequent ligation and amplification procedures (see, e.g., Frank et al. (1987), Methods Enzymol. 154: 221-249).

It has now been found that the humanized antibody referred to supra, i.e., BIBH 1, has a surprisingly short half life, making it exceptionally useful as a therapeutic agent, since the issues raised by a long half life are averted.

The antibody proteins to be used according to the invention provide a highly specific tool for targeting therapeutic agents to the FAPα antigen. Therefore, in a further aspect, the invention relates to antibody proteins according to the invention, wherein said antibody protein is conjugated to a therapeutic agent. Of the many therapeutic agents known in the art, therapeutic agents selected from the group consisting of radioisotopes, toxins, toxoids, inflammatogenic agents, enzymes, antisense molecules, peptides, cytokines, and chemotherapeutic agents are preferred. Among the radioisotopes, gamma, beta and alpha-emitting radioisotopes may be used as a therapeutic agent. β-emitting radioisotopes are preferred as therapeutic radioisotopes. ¹⁸⁶Rhenium, ¹⁸⁸Rhenium, ¹³¹Iodine, and ⁹⁰Yttrium have been proven to be particularly useful β-emitting isotopes to achieve localized irradiation and destruction of malignant tumor cells. Therefore, radioisotopes selected from the group consisting of ¹⁸⁶Rhenium, ¹⁸⁸Rhenium, ¹³¹Iodine and ⁹⁰Yttrium are particularly preferred as therapeutic agents conjugated to the antibody proteins of the invention. Most preferred is ¹³¹Iodine. For example, for the radioiodination of an antibody of the invention, a method as disclosed in WO 93/05804 may be employed.

Thus, a more preferred aspect of the present invention is the use of an antibody protein according to the invention, wherein said therapeutic agent is a therapeutic agent selected from the group consisting of radioisotopes, pro-drugs and chemotherapeutic agents.

Another preferred aspect of the present invention is the use of an antibody according to the invention, wherein said radioisotope is selected from the group consisting of ¹⁸⁶Rhenium, ¹⁸⁸Rhenium, ¹³¹Iodine and ⁹⁰Yttrium.

A most preferred aspect of the present invention is a pharmaceutical composition according the invention, wherein said radioisotope is ¹³¹Iodine.

Another more preferred aspect of the present invention is the use of an antibody according to the invention, wherein said radioisotope is a γ-emitting radioisotope. Another most preferred aspect of the present invention is the use of an antibody according to the invention, wherein said radioisotope is 125I.

Hence, a most important aspect of the invention is a method for treating pathological conditions such as cancer, epithelial carcinomas in particular, by administering a therapeutically effective amount of an antibody such as a humanized antibody, which specifically binds to FAPα. Any of the antibody molecules as set out supra may be used in said method according to the invention.

It should be noted that when “antibody” is used herein, this refers to the whole antibody, as well as any and all fragments of the antibody which possess the ability to bend to the target antigen. It was noted, supra, that the antibody is produced recombinantly, and one of ordinary skill in the art will be familiar with how to engineer production of said antibody fragments which retain the ability to bend to the target antigen.

The antibody is preferably formulated as a solution of the antibody in a physiologically acceptable solvent, e.g. an aqueous solution between pH 7 and 8. The pH may be stabilized by a pharmaceutically acceptable buffer. The solution may also contain further stabilizing agents like a detergent like Tween 20, serum albumin, or ascorbic acid. For example, the antibody may be solved in an aqueous buffer like phosphate buffered saline containing 0.02% Tween 20, pH 7.4±0.2, or may be solved in saline (0.9% NaCl) containing 5% human serum albumin. In one embodiment, the antibody will be at an end concentration of 0.1 to 10 mg of antibody per mL solution, more preferably 0.5 to 2 mg/mL, more preferably about 1 mg/mL.

Therefore, preferably an antibody protein that contains the variable region of the light chain selected from the group as set forth in SEQ ID NO: 2, SEQ ID NO: 4, or SEQ ID NO: 6 may be administered to the patient in need thereof.

Preferably an antibody protein, characterized in that the variable region of the light chain is encoded by a nucleotide sequence selected from the group as set forth in SEQ ID NO: 1, SEQ ID NO: 3, or SEQ ID NO: 5 may be administered to the patient in need thereof.

Preferably an antibody protein containing a variable region of the heavy chain as set forth in any one of SEQ ID NOs: 8, 10, 12, 14, or 16, may be administered to the patient in need thereof.

Preferably an antibody protein characterized in that the variable region of the heavy chain is encoded by a nucleotide sequence as set forth in any one of SEQ ID NOs: 7, 9, 11, 13, or 15, may be administered to the patient in need thereof.

Preferably an antibody protein containing a constant region of the light chain as set forth in SEQ ID NO: 18 may be administered to the patient in need thereof.

Preferably an antibody protein containing a constant region of the heavy chain as set forth in SEQ ID NO: 20 may be administered to the patient in need thereof.

Preferably an antibody protein characterized in that the constant region of the light chain is encoded by a nucleotide sequence as set forth in SEQ ID NO: 17 may be administered to the patient in need thereof. Preferably an antibody protein characterized in that the constant region of the heavy chain is encoded by a nucleotide sequence as set forth in SEQ ID NO: 19 may be administered to the patient in need thereof.

More particularly, the antibody is the antibody referred to as BIBH 1, discussed supra, and disclosed in WO 99/57151, incorporated by reference. Hence, in a most preferred embodiment an antibody protein containing the variable region of the light chain as set forth in SEQ ID NO: 2 and the variable region of the heavy chain as set forth in SEQ ID NO: 12 may be administered to the patient in need thereof. Most preferably, this antibody protein additionally contains the constant region of the light chain as set forth in SEQ ID NO: 20 and the constant region of the heavy chain as set forth in SEQ ID NO: 22.

Hence, one aspect of the invention is a method for treating a subject with cancer, wherein the cancer tumor or cancer cells of said subject express an antigen specifically bound by antibody BIBH 1. The treatment involves administering, inter alia, an amount of labeled BIBH 1 antibody to said subject that is sufficient to have a therapeutic effect on said subject. The artisan is familiar with the type of label that can be used on humanized antibodies, such as chemotherapeutic agents, approtactic agents, agents which inhibit DNA expression and, most particularly, radioactive agents. Of the many therapeutic agents known in the art, therapeutic agents selected from the group consisting of radioisotopes, inflammatogenic agents, enzymes, antisense molecules, peptides, cytokines, and chemotherapeutic agents are preferred. Thus, in a further aspect, an antibody protein according to the invention may be administered to the patient in need thereof, wherein said antibody protein is conjugated to a therapeutic agent. Among the radioisotopes, gamma, beta-, and alpha-emitting radioisotopes may be used as a therapeutic agent. β-emitting radioisotopes are preferred as therapeutic radioisotopes. Among the type of such radiolabels which can be used are ¹³¹Iodine, ¹²⁵Iodine, ⁹⁰Yttrium, ¹⁸⁶Rhenium, ¹⁸⁸Rhenium, various isotypes of cobalt, indium, and other radioactive materials. See, e.g., WO 93/05804 incorporated by reference, for protocols for radiolabeling antibodies. ¹⁸⁶Rhenium, ¹⁸⁸Rhenium, ¹³¹Iodine and ⁹⁰Yttrium have been proven to be particularly useful β-emitting isotopes to achieve localized irradiation and destruction of malignant tumor cells. Therefore, radioisotopes selected from the group consisting of ¹⁸⁶Rhenium, ¹⁸⁸Rhenium, ¹³¹Iodine and ⁹⁰Yttrium are particularly preferred as therapeutic agents conjugated to the antibody proteins of the invention.

Most preferably, said antibody is labeled with ¹³¹Iodine.

Cancer includes any disease associated with malignant growth such as solid tumors, sarcomas and leukemias. A necessary precondition for such diseases is the expression of FAPα in the tumor-associated stroma. Cancer according to the invention includes, but is not limited to:

1) The treatment of epithelial carcinomas including breast, lung, colorectal, head and neck, pancreatic, ovarian, bladder, gastric, skin, endometrial, ovarian, testicular, esophageal, prostatic and renal origin;

2) Bone and soft-tissue sarcomas: Osteosarcoma, chondrosarcoma, fibrosarcoma, malignant fibrous histiocytoma (MFH), leiomyosarcoma;

3) Hematopoietic malignancies: Hodgkin's and non-Hodgkin's lymphomas;

4) Neuroectodermal tumors: Peripheral nerve tumors, astrocytomas, melanomas; Mesotheliomas.

Examples for cancerous disease states associated with solid tumors include, but are not limited to: colorectal cancer, non-small cell lung cancer, breast cancer, head and neck cancer, ovarian cancer, lung cancer, bladder cancer, pancreatic cancer and metastatic cancers of the brain.

The invention further relates to a method of treating cancer, wherein the pharmaceutical composition according the invention is administered once to several times to an individual in need thereof, said antibody protein selectively binds to FAPα, the tumor cells are destroyed by the radioisotope linked to the antibody protein and by the chemotherapeutic agent, and the therapeutic success is monitored. The method of treating tumors as described above may be effected in vitro or in vivo. Cancer is defined as set out above.

Thus, another most preferred embodiment is the method according the invention, wherein said cancer is selected from the group consisting of colorectal cancer, non-small cell lung cancer, breast cancer, head and neck cancer, ovarian cancer, lung cancer, bladder cancer, pancreatic cancer and metastatic cancers of the brain.

The method according to the invention comprises administering the antibody according to the invention by any route suitable as determined by the artisan. Examples of such routes are described in detail infra. Thus, preferably the method according to the invention comprises administering said antibody or antibody fragment intravenously.

Said antibody molecule to be administered or pharmaceutical composition or medicament may further comprise a pharmaceutically acceptable carrier or excipient.

A pharmaceutically acceptable carrier can contain physiologically acceptable compounds that act, for example, to stabilize or to increase the absorption of an AMPA glutamate receptor agonist, antagonist or modulator. Such physiologically acceptable compounds include, for example, carbohydrates, such as glucose, sucrose or dextrans, antioxidants, such as ascorbic acid or glutathione, chelating agents, low molecular weight proteins or other stabilizers or excipients (see also, e.g., Remington's Pharmaceutical Sciences (1990)). One skilled in the art would know that the choice of a pharmaceutically acceptable carrier, including a physiologically acceptable compound, depends, for example, on the route of administration of the composition.

In an animal or human body, it can prove advantageous to apply antibody or medicament as described above via an intravenous or other route, e.g. systemically, locally or topically to the tissue or organ of interest, depending on the type and origin of the disease or problem treated, e.g. a tumor. For example, a systemic mode of action is desired when different organs or organ systems are in need of treatment as in e.g. systemic autoimmune diseases, or allergies, or transplantations of foreign organs or tissues, or tumors that are diffuse or difficult to localize. A local mode of action would be considered when only local manifestations of neoplastic or immunologic action are expected, such as, for example local tumors.

The antibody compositions of the present invention may be applied by different routes of application known to the expert, notably intravenous injection or direct injection into target tissues. For systemic application, the intravenous, intravascular, intramuscular, intraarterial, intraperitoneal, oral, or intrathecal routes are preferred. A more local application can be effected subcutaneously, intracutaneously, intracardially, intralobally, intramedullarly, intrapulmonarily or directly in or near the tissue to be treated (connective-, bone-, muscle-, nerve-, epithelial tissue). Therapy with the labeled antibody may be the entire therapeutic regime, or it may be a part of a regime which includes, e.g., chemotherapy, therapy with an additional antibody or other forms of standard therapeutic approaches to cancer.

Depending on the desired duration and effectiveness of the treatment, pharmaceutical antibody compositions may be administered once or several times, also intermittently, for instance on a daily basis for several days, weeks or months and in different dosages.

For preparing suitable antibody preparations for the applications described above, the expert may use known injectable, physiologically acceptable sterile solutions. For preparing a ready-to-use solution for parenteral injection or infusion, aqueous isotonic solutions, such as e.g. saline or corresponding plasma protein solutions are readily available. The antibody compositions or pharmaceutical compositions or medicaments may be present as lyophylisates or dry preparations, which can be reconstituted with a known injectable solution directly before use under sterile conditions, e.g. as a kit of parts. The final preparation of the antibody compositions pharmaceutical compositions or medicaments of the present invention are prepared for injection, infusion or perfusion by mixing purified antibodies according to the invention with a sterile physiologically acceptable solution, that may be supplemented with known carrier substances or/and additives (e.g. serum albumin, dextrose, sodium bisulfite, EDTA).

The amount of the antibody applied depends on the nature of the disease. It will be understood that the dose that is administered to the subject may vary, depending on parameters which include the severity of the cancer, the age, weight, and other intrinsic characteristics of the patient being treated, the overall health of the patient, prior forms of treatment, and so forth.

In cancer patients, the applied dose of a ‘naked’ antibody may be between 0.1 and 100 mg/m², preferably between 5 and 50 mg/m² per application. For radiolabeled antibodies, e.g. with iodine-131, the maximally tolerated dose (MTD) has to be determined which must not be exceeded in therapeutic settings. Application of radiolabeled antibody to cancer patients may then be carried out by repeated (monthly or weekly) intravenous infusion of a dose which is at or below the MTD (see, e.g., Welt et al. (1994) J. Clin. Oncol. 12: 1193-1203).

The dose of radioactivity applied to the patient per administration has be high enough to be effective, but must be below the dose limiting toxicity (DLT). In general, a sufficiently well tolerated dose below DLT will be considered “maximum” tolerated dose,” or “MTD.” The expert knows how to determine the MTD; see, e.g., Welt et al., J Clin Oncol 12(8), 1561-1571 (1994); Welt et al (1994) J Clin Oncol 12:1193-1203 (1994). Furthermore, the applied radioactivity dose will be in accordance with the guidelines outlined below. In general, the radioactivity dose per administration will be between 30 and 75 mCi/m² body surface area (BSA).

Multiple administrations are preferred, generally at weekly intervals; however, radiolabeled materials should be administered at longer intervals, i.e., 4-24 weeks apart, preferable 12-20 weeks apart. The artisan may choose, however, to divide the administration into two or more applications, which may be applied shortly after each other, or at some other predetermined interval ranging, e.g. from 1 day to 1 week. As will be seen, infra, the preferred label for the antibody is ¹³¹I.

Hence, another preferred method according to the invention comprises administering said antibody or antibody fragment at a dose of from about 5 mg/m² to about 50 mg/m² body surface area.

Yet another preferred method according to the invention comprises administering said antibody or antibody fragment at a dose of from about 10 mg/m² to about 40 mg/m² body surface area.

Yet another preferred method according to the invention comprises administering said antibody or antibody fragment at a dose of from about 10 mg/m² to about 30 mg/m² body surface area.

Yet another preferred method according to the invention comprises administering said antibody at a dose of from about 20 mg/m² to about 30 mg/m² body surface area.

Yet another most preferred method according to the invention comprises administering said antibody or antibody fragment at a dose of about 25 mg/m² body surface area.

Yet another most preferred method according to the invention comprises administering said antibody or antibody fragment at a dose of about 50 mg/m² body surface area.

Another preferred method according to the invention comprises administering said antibody or antibody fragment in combination with at least one additional therapeutic agent.

As set out supra, preferred are radioisotopes. Upon functional linkage of the antibody with the radioisotope, said antibody-radioisotope conjugate has therapeutic utility due to the radiation.

To achieve an antibody preparation allowing application of the desired amount of antibody and the desired radioactivity dose, the specific radioactivity of the antibody will have to be adjusted accordingly. The expert knows how to achieve a desired specific radioactivity in a radiolabeling process.

The radioactivity of the conjugate is expressed as specific activity. Hence, in another preferred method according to the invention, said antibody has specific activity of from about 0.5 to about 15 mCi/mg.

In another preferred method according to the invention, said specific activity is from about 0.5 to about 14 mCi/mg.

In another preferred method according to the invention, said specific activity is from about 1 to about 10 mCi/mg.

In another preferred method according to the invention, said specific activity is from about 1 to about 5 mCi/mg.

In another preferred method according to the invention, said specific activity is from 2 to 6 mCi/mg.

In another preferred method according to the invention, said specific activity is from about 1 to about 3 mCi/mg.

Another preferred method according to the invention comprises administering said antibody in an aqueous solution at pH of from about 7 to about 8, and at a concentration of from about 0.5 to about 2.0 mg/mL.

Another important embodiment of the present invention is a pharmaceutical composition comprising an antibody molecule or antibody derivative as defined supra.

Therefore, preferably said pharmaceutical composition comprises an antibody protein that contains the variable region of the light chain selected from the group as set forth in SEQ ID NO: 2, SEQ ID NO: 4 or SEQ ID NO: 6.

Preferably said pharmaceutical composition comprises an antibody protein, characterized in that the variable region of the light chain is encoded by a nucleotide sequence selected from the group as set forth in SEQ ID NO: 1, SEQ ID NO: 3 or SEQ ID NO: 5.

Preferably said pharmaceutical composition comprises an antibody protein containing a variable region of the heavy chain as set forth in any one of SEQ ID NOs: 8, 10, 12, 14, 16.

Preferably said pharmaceutical composition comprises an antibody protein characterized in that the variable region of the heavy chain is encoded by a nucleotide sequence as set forth in any one of SEQ ID NOs: 7, 9, 11, 13, 15.

Preferably said pharmaceutical composition comprises an antibody protein containing a constant region of the light chain as set forth in SEQ ID NO: 18.

Preferably said pharmaceutical composition comprises an antibody protein containing a constant region of the heavy chain as set forth in SEQ ID NO: 20.

Preferably said pharmaceutical composition comprises an antibody protein characterized in that the constant region of the light chain is encoded by a nucleotide sequence as set forth in SEQ ID NO: 17. Preferably an antibody protein characterized in that the constant region of the heavy chain is encoded by a nucleotide sequence as set forth in SEQ ID NO: 19.

More particularly, said pharmaceutical composition comprises the antibody referred to as BIBH 1, discussed supra, and disclosed in WO 99/57151, incorporated by reference. Hence, in a most preferred embodiment said pharmaceutical composition comprises an antibody protein containing the variable region of the light chain as set forth in SEQ ID NO: 2 and the variable region of the heavy chain as set forth in SEQ ID NO: 12. Most preferably, said antibody protein comprised in said pharmaceutical composition comprises additionally contains the constant region of the light chain as set forth in SEQ ID NO: 20 and the constant region of the heavy chain as set forth in SEQ ID NO: 22.

Said antibody molecule may also be functionally linked to a therapeutic agent as defined supra. Thus in a further aspect, said antibody protein comprised in said pharmaceutical composition is conjugated to a therapeutic agent. Of the many therapeutic agents known in the art, therapeutic agents selected from the group consisting of radioisotopes, toxins, toxoids, inflammatogenic agents, enzymes, antisense molecules, peptides, cytokines, and chemotherapeutic agents are preferred. Among the radioisotopes, gamma, beta and alpha-emitting radioisotopes may be used as a therapeutic agent. β-emitting radioisotopes are preferred as therapeutic radioisotopes. ¹⁸⁶Rhenium, ¹⁸⁸Rhenium, ¹³¹Iodine and ⁹⁰Yttrium have been proven to be particularly useful β-emitting isotopes to achieve localized irradiation and destruction of malignant tumor cells. Therefore, radioisotopes selected from the group consisting of ¹⁸⁶Rhenium, ¹⁸⁸Rhenium, ¹³¹Iodine and ⁹⁰Yttrium are particularly preferred as therapeutic agents conjugated to the antibody proteins of the invention.

Most preferably, said antibody is labeled with ¹³¹Iodine.

The definitions and explanations provided for the method of pathological disorders (supra), in particular cancer treatment, application route, also apply to the pharmaceutical composition comprising an antibody molecule or antibody derivative according to the invention.

Preferably, the pharmaceutical composition comprising an antibody molecule or antibody derivative according to the invention as defined supra is administered in an application selected from the group of intravenous, intravascular, intramuscular, intraarterial, intraperitoneal, oral, intrathecal injection, direct injection into or near the target tissues, subcutaneous, intracutaneous, intracardial, intralobal, intramedullary, intrapulmonary application.

The manner in which the antibody is administered may vary, but a single injection or infusion administration, preferably intravenous administration, such as by bolus or infusion, is preferred. Thus, most preferably is the pharmaceutical composition comprising an antibody molecule or antibody derivative according to the invention as defined supra is administered intravenously.

Preferably is the pharmaceutical composition comprising an antibody molecule or antibody derivative according to the invention as defined supra further comprises a pharmaceutically acceptable carrier or excipient.

Preferably also is the pharmaceutical composition comprising an antibody molecule or antibody derivative according to the invention as defined supra, wherein the amount of antibody or antibody derivative per application is between 0.1 and 100 mg/m², preferably between 5 and 50 mg/m², preferably 10 mg/m² to about 40 mg/m², preferably 10 mg/m² to about 30 mg/m², also preferably 20 mg/m² to about 30 mg/m², and most preferably about 25 mg/m² body surface area. Also most preferred is about 50 mg/m² body surface area.

Preferably also is the pharmaceutical composition comprising an antibody molecule or antibody derivative according to the invention as defined supra, wherein the pharmaceutical composition comprises at least one additional therapeutic agent.

Preferred also is the pharmaceutical composition comprising an antibody molecule or antibody derivative conjugated to a radioisotope according to the invention as defined supra, wherein the antibody or antibody derivative has specific activity of from about 0.5 to about 15 mCi/mg, or from about 0.5 to about 14 mCi/mg, preferably about 1 to about 10 mCi/mg, preferably about 1 to about 5 mCi/mg, and most preferably 2 to 6 mCi/mg or 1 to 3 mCi/mg.

Preferred also is the pharmaceutical composition comprising an antibody molecule or antibody derivative conjugated to a radioisotope according to the invention as defined supra, wherein said antibody or antibody derivative is in an aqueous solution at pH of from about 7 to about 8, and at a concentration of from about 0.5 to about 2.0 mg/mL.

Another preferred embodiment of the present invention is the use of an antibody molecule or antibody derivative as defined supra in the manufacture of a medicament for treatment of cancer.

Therefore, preferably said antibody protein contains the variable region of the light chain selected from the group as set forth in SEQ ID NO: 2, SEQ ID NO: 4 or SEQ ID NO: 6.

Preferably, said antibody protein is characterized in that the variable region of the light chain is encoded by a nucleotide sequence selected from the group as set forth in SEQ ID NO: 1, SEQ ID NO: 3 or SEQ ID NO: 5.

Preferably, said antibody protein is containing a variable region of the heavy chain as set forth in any one of SEQ ID NOs: 8, 10, 12, 14, 16.

Preferably, said antibody protein is characterized in that the variable region of the heavy chain is encoded by a nucleotide sequence as set forth in any one of SEQ ID NOs: 7, 9, 11, 13, 15.

Preferably, said antibody protein is containing a constant region of the light chain as set forth in SEQ ID NO: 18.

Preferably, said antibody protein is containing a constant region of the heavy chain as set forth in SEQ ID NO: 20.

Preferably, said antibody protein is characterized in that the constant region of the light chain is encoded by a nucleotide sequence as set forth in SEQ ID NO: 17. Preferably an antibody protein characterized in that the constant region of the heavy chain is encoded by a nucleotide sequence as set forth in SEQ ID NO: 19.

More particularly, the invention relates to the use of the antibody referred to as BIBH 1, discussed supra, and disclosed in WO 99/57151, incorporated by reference in the manufacture of a medicament for treatment of cancer. Hence, in a most preferred embodiment, said antibody protein is containing the variable region of the light chain as set forth in SEQ ID NO: 2 and the variable region of the heavy chain as set forth in SEQ ID NO: 12. Most preferably, said antibody protein comprised in said pharmaceutical composition comprises additionally contains the constant region of the light chain as set forth in SEQ ID NO: 20 and the constant region of the heavy chain as set forth in SEQ ID NO: 22.

Said antibody molecule may also be functionally linked to a therapeutic agent as defined supra. Thus in a further aspect, said use of an antibody protein in the manufacture of a medicament of cancer relates to an antibody protein which is conjugated to a therapeutic agent. Of the many therapeutic agents known in the art, therapeutic agents selected from the group consisting of radioisotopes, toxins, toxoids, inflammatogenic agents, enzymes, antisense molecules, peptides, cytokines, and chemotherapeutic agents are preferred.

Among the radioisotopes, gamma-, beta-, and alpha-emitting radioisotopes may be used as a therapeutic agent. β-emitting radioisotopes are preferred as therapeutic radioisotopes. ¹⁸⁶Rhenium, ¹⁸⁸Rhenium, ¹³¹Iodine and ⁹⁰Yttrium have been proven to be particularly useful β-emitting isotopes to achieve localized irradiation and destruction of malignant tumor cells. Therefore, radioisotopes selected from the group consisting of ¹⁸⁶.Rhenium, ¹⁸⁸Rhenium, ¹³¹Iodine and ⁹⁰Yttrium are particularly preferred as therapeutic agents conjugated to the antibody proteins of the invention.

Most preferably, said antibody is labeled with ¹³¹Iodine.

Cancer is any cancer as defined supra. The definitions and explanations provided for the method of pathological disorders (supra), in particular cancer treatment, also apply to the use of an antibody molecule or antibody derivative according to the invention in the manufacture of a medicament for treatment of cancer.

Thus, another most preferred embodiment is the use of an antibody molecule or antibody derivative according to the invention as defined supra in the manufacture of a medicament for treatment of cancer, wherein said cancer is selected from the group consisting of colorectal cancer, non-small cell lung cancer, breast cancer, head and neck cancer, ovarian cancer, lung cancer, bladder cancer, pancreatic cancer and metastatic cancers of the brain.

Preferably is the use of an antibody molecule or antibody derivative according to the invention as defined supra in the manufacture of a medicament for treatment of cancer for an application selected from the group of intravenous, intravascular, intramuscular, intraarterial, intraperitoneal, oral, intrathecal injection, direct injection into or near the target tissues, subcutaneous, intracutaneous, intracardial, intralobal, intramedullary, intrapulmonary application.

Most preferably is the use of an antibody molecule or antibody derivative according to the invention as defined supra in the manufacture of a medicament for treatment of cancer for intravenous application.

Preferably is the use of an antibody molecule or antibody derivative according to the invention as defined supra in the manufacture of a medicament for treatment of cancer, wherein said medicament further comprises a pharmaceutically acceptable carrier or excipient.

Preferably also is the use of an antibody molecule or antibody derivative according to the invention as defined supra in the manufacture of a medicament for treatment of cancer, wherein the amount of antibody or antibody derivative per application is between 0.1 and 100 mg/m², preferably between 5 and 50 mg/m², preferably 10 mg/m² to about 40 mg/m², preferably 10 mg/m² to about 30 mg/m², also preferably 20 mg/m² to about 30 mg/m², and most preferably about 25 mg/m² body surface area. Also most preferred is an antibody or antibody derivative dose of about 50 mg/m² body surface area.

Preferably also is the use of an antibody molecule or antibody derivative according to the invention as defined supra in the manufacture of a medicament for treatment of cancer, wherein the medicament comprises at least one additional therapeutic agent.

Preferred also is the use of an antibody molecule or antibody derivative conjugated to a radioisotope according to the invention as defined supra in the manufacture of a medicament for treatment of cancer, wherein the antibody or antibody derivative has specific activity of from about 0.5 to about 15 mCi/mg, or from about 0.5 to about 14 mCi/mg, preferably about 1 to about 10 mCi/mg, preferably about 1 to about 5 mCi/mg, and most preferably 2 to 6 mCi/mg or 1 to 3 mCi/mg.

Preferred also is the use of an antibody molecule or antibody derivative conjugated to a radioisotope according to the invention as defined supra in the manufacture of a medicament for treatment of cancer, wherein said antibody or antibody derivative is in an aqueous solution at pH of from about 7 to about 8, and at a concentration of from about 0.5 to about 2.0 mg/mL.

The following examples serve to further illustrate the present invention; but the same should not be construed as limiting the scope of the invention disclosed herein.

### EXAMPLE 1

A humanized antibody against FAPα was prepared, in accordance with the disclosure of WO 99/57151, incorporated by reference. As noted therein, this is a CDR grafted, humanized antibody against human FAPα. The antibody is BIBH 1. This antibody was used in an in vivo trial, as follows.

Twenty six patients (15 male, 11 female, mean age 59.9 years, age range 41-81 years) were entered into the trial. Twenty patients suffered from colorectal carcinoma, and six from non-small cell lung carcinoma.

The patients were entered into four dosage tiers, receiving 5, 10, 25, or 50 mg/m² of BIBH 1. A treatment cycle consisted of 12, weekly intravenous infusions over a 60 minute period. The dose administered at weeks 1, 5 and 9 were labeled with 8-10 mCi of ¹³¹I, while the remaining 9 doses were unlabelled. Serum samples were taken from patients at weekly intervals, in order to determine if HAHA had occurred. The analyses of these samples is described infra, in the additional examples. At the conclusion of a 12 week cycle, additional cycles were undertaken with some of the patients.

The samples were analyzed to determine the whole body clearance (biologic T½), and patients were also studied to determine biodistribution patterns, normal organ dosimetry, tumor dosimetry and red marrow doses, using standard methods.

One episode of dose limiting toxicity was observed, so maximum tolerated dose could not be reached in this patient. Adverse effects due to the antibody were observed in 6 patients, and 4 of these patients were removed from the study, due to the clinical immune response observed, which included rigors, chills, and flushing.

Whole body clearance of ¹³¹I-BIBH 1 was calculated to be (mean±SD) 104.9±40.9, 127.6±40.9, 137.7±34.6 and 179.8±53.5 hours for the 5, 10, 25 and 50 mg/m² dose levels. When first infusion only values were analyzed, which removed any confounding effect of HAHA, whole body clearance values were calculated to be (mean±SD) 98.7±23.5, 124.8±26.9, 137.1±16.4 and 166.6±49.7 hours for the 5, 10, 25 and 50 mg/m² dose levels respectively. Statistically, there was a strong linear relation between T½ and BIBH 1 dose (p<0.001); T½ increased with dose. This linear relationship was significant, regardless of how T½ was summarized for each patient (mean, minimum, maximum levels, week 1, week 5, and week 9). Biologic T½ values were consistent within patients between infusions when HAHA was not present. The development of HAHA was associated with faster whole body clearance, compared to infusion 1 datasets of each patient, and was observed in 5/25 patients with evaluable image datasets, and 6/64 evaluable ¹³¹I-BIBH 1 infusions.

The biodistribution pattern was consistent with blood pool activity, with no normal organ increased uptake for the first infusion of ¹³¹I-BIBH 1 in all patients at all dose levels. Tumor uptake was evident by as early as 24-48 hours after infusion. The development of HAHA, when present, was associated with faster blood pool and normal organ clearance, and reduced targeting of tumor, compared to the first infusion in all patients. In one patient (Pt. 102, infusion 5) was HAHA associated with stable liver uptake.

With respect to whole body and normal organ dosimetry, the results for individual organs and whole body doses were similar within the 5, 10 and 25 mg/m² dose levels, with higher values at the 50 mg/m² dose level, most likely as a result of the longer serum half life at this dose level. Target organs with the highest dose were lungs, liver, spleen and kidney, consistent with the blood pool appearance on gamma camera images. An apparent trend was observed for increasing mean±SD organ dose for lung, liver, spleen and kidney with increasing ¹³¹I-BIBH 1 dose, but this linear trend was not apparent or significant when individual patient data were examined for linear relationships. No marked differences were observed between ¹³¹I-BIBH 1 dose levels for liver and lung organ dosimetry, but the 50 mg/m² kidney and spleen organ doses were significantly greater than those of the 10 and 5 mg/m² doses, respectively (P<0.05). The total body effective dose equivalent mean±SD values were 1.8±0.4, 1.8±0.2, 2.1±0.2, and 2.4±0.5 rem/mCi for the first infusion at the 5, 10, 25 and 50 mg/m² dose levels. No unexpectedly high values were observed for any normal organ. The development of HAHA was associated with faster clearance, and reduced total dose to normal organs (exception Pt. 102 with stable liver uptake at week 5).

Red marrow absorbed dose calculations showed a similar mean±SD values for all ¹³¹I-BIBH 1 infusions for the 5, 10 and 25 mg/m² dose levels (1.50±0.59, 1.56±0.52, and 1.47±0.38 rad/mCi). Red marrow absorbed dose was significantly higher at the 50 mg/m² dose level (2.47±0.46 rad/mCi) than the 5, 10 and 25 mg/m² dose levels (P<0.001, P<0.002, P<0.001, respectively), in keeping with the prolonged serum clearance in the 50 mg/m² patients. When first infusion only values were analyzed, which removed any confounding effect of HAHA, red marrow dose values were calculated to be (mean±SD) 1.62±0.37, 1.59±0.43, 1.58±0.32 and 2.34±0.55 hours for the 5, 10, 25 and 50 mg/m² dose levels respectively. HAHA, when present, was associated with a reduced red marrow dose in all patients.

These data suggest that the half life for doses up to 25 mg/m² in the circulation is remarkably short, rendering the antibody suitable for in vivo administration at lower doses, as compared to the higher dose of 50 mg/m².

The clearance of the antibody, as elaborated supra, is such that it is cleared slowly enough to permit effective tumor uptake, and fast enough to reduce the risk of side effects significantly, while staying well within the maximum tolerated dose.

### EXAMPLE 2A

This example details a smaller study, using 17 patients, who were treated using the same protocol described in example 1. Nine of 17 patients completed at least one cycle of treatment. Due to progressive disease, or other events, eight of the patients received less than one cycle. One of the patients in the group of nine has actually received 5, complete cycles, and a second patient is completing a second cycle. Analysis of these results follows.

The HAHA effect was alluded to in example 1. This example explains how this effect was studied.

Blood samples (5 mL) were collected from each patient every week. The blood sample was taken from the arm of the patient not used for the BIBH 1 infusion therapy. Blood was allowed to clot, at room temperature. The resulting serum (about 2 mL per sample), was centrifuged, pipetted into 0.5 mL to 1 mL aliquots into cryotubes, and frozen. In addition to the twelve weeks of treatment, samples were obtained in the 13^{th} week, and a subsequent follow up visit.

Samples were analyzed first by ELISAs, and then via BIACORE®. In the ELISAs, samples of BIBH 1 were immobilized to microtiter plates, and then serum samples were added, to permit any anti-BIBH 1 antibodies to bind thereto. After contact, serum components were removed by washing, and biotin labeled BIBH 1 was added. The labeled antibody was incubated, complexes were washed, and any biotinylated antibodies bound thereto were identified with streptavidin—horseradish peroxidase complexes. Standard, chromogenic substrates were used to detect the enzyme. Colored product was measured photometrically. The amount of color increased with increasing concentrations of anti-BIBH 1 antibodies in the samples. Antibody concentration corresponding to measured color was calculated via data fitting of the non-linear standard curve, and the assay was validated using a mouse anti-idiotype anti-BIBH 1 antibody.

The measurement range of anti-BIBH 1 antibodies in the HAHA ELISA ranged from 20-2000 ng/mL, after a 1:10 dilution of serum.

Six out of the seventeen patients had measurable positive serum HAHA levels at the limit of detection (20 ng/mL), after 2-8 infusions of the antibody.

While the HAHA response seemed to be associated with pharmacokinetic changes during repeated dosing, only one example of treatment associated toxicity occurred, in only one patient.

A total of 292 samples were analyzed, and HAHA was measurable at baseline before 20 infusions, yielding a per infusion HAHA incidence of about 10% HAHA decreased from 4/7 patients in the 5 mg/m² group, to 0/3 in the 50 mg/m² group.

### EXAMPLE 2B

This example details a study, using 26 patients, who were treated using the same protocol described in example 1. Analysis of these results follows. HAHA were analyzed by ELISA and BIACORE® as described in Example 2A.

Eight out of 25 patients who received at least 2 infusions exhibited positive anti-BIBH 1 responses at some time during the dosage regimen both in the ELISA and BIACORE® assays, corresponding to a HAHA incidence of 32%. The HAHA responses increased with continued dosing of the eight positive patients. The earliest and latest times at which anti-BIBH 1 serum reactivity first became detectable were directly before the 3^{rd} infusion and directly before the 9^{th} infusion, respectively (measured by BIACORE®). Thus, the time of onset of HAHA development was quite variable.

The maximum concentration of HAHA attained during the dosage regimen varied considerably between patients. Expressed in anti-idiotype concentration equivalents as measured in the ELISA, the highest peak HAHA in the patients with a complete sampling schedule was 17.9 μg/mL at 7 days after the last infusion, and the lowest was 0.248 μg/mL at 30 days after the last infusion.

The incidence and magnitude of the HAHA response correlated well with alterations in pharmacokinetics. However, treatment associated toxicity that might have been related to BIBH 1 occurred in only four of the eight patients with HAHA development. There did not appear to be a correlation between the magnitude of the HAHA response and adverse events. One patient experienced adverse events considered to be related to BIBH 1 but did not develop HAHA.

There was a reduced incidence of HAHA with increasing dose. 4/7 patients developed HAHA in the 5 mg/m² group compared with ⅙ at 10 mg/m², 2/6 at 25 mg/m² and ⅙ at 50 mg/m². It is possible that higher serum concentrations of BIBH 1 lead to a suppression of the immune response.

### EXAMPLE 3

The BIACORE® method relies on instrumentation and methodologies described in, e.g. Jönsson, et al., Advances in Biosensors 2:291-336 (1992) and Malmqvist, et al., Nature 361:186-187 (1993), both of which are incorporated by reference. Essentially the instrumentation permits detection of biomolecules and monitoring of binding events between two molecules in real time, without the use of labels.

The instrument used in these assays includes four, flow through cells in sequence, connected to a sensor chip. Two of these cells were used. In the first cell, a fixed amount of BIBH 1 was covalently immobilized to a sensor surface, and a comparable amount of humanized A33 monoclonal antibody was immobilized to the surface of the second cell. (This humanized A33 mAb was used because its framework is similar to BIBH 1, but the antibody does not bind FAPα).

The design of the experiment permitted better characterization of the HAHA response, because if serum components bound to both surfaces an anti-isopic response would be indicated, whereas binding to BIBH 1 only would indicate an anti-idiotypic response.

Samples were diluted 1:100 prior to analysis and then 10 μl samples were passed over the sensor surface, using a microfluidic system, for one minute. Any non-specifically bound components were washed out via injection of wash buffer. Any differences between the resulting signal, and blank value before injection of sample, indicated amount of anti-BIBH 1 response. Of the samples that exhibited a positive HAHA reactivity in the BIACORE® assay, no serum showed a significant reactivity against the immobilized control antibody huAb A33 This indicates a mainly anti-idiotypic immune response of the patients with positive HAHA response.

IgG was selectively removed from the serum samples using protein A- or protein G-Sepharose precipitation prior to BIACORE® analysis. After the protein A/G precipitation, BIACORE® reactivity was abolished in sera of all eight patients. The results from these assays indicated that the HAHA response was anti-idiotypic and IgG (rather than IgM) in nature.

### EXAMPLE 4

This example describes studies on the pharmacokinetics of the antibody. As was indicated, supra, ¹³¹I labeled antibody was administered to subjects at periodic intervals, i.e., weeks 1, 5, and 9. Either 7 or 8 total samples were taken at each of these intervals. The “TOPFIT” program, described by Heinzel, et al, TopFit Version 2.0: Pharmacokinetic and Pharmacodynamic Data Analysis System for the PC, Gustav Fischer Verlag: Stuttgart 1993, incorporated by reference, was used. Serum radioactivity time profiles which were obtained in weeks 1, 5, and 9 were analyzed using standard, non-compartmental methods, in accordance with Gibaldi, Biopharmaceutics and Clinical Pharmacokinetics, Philadelphia: Lea & Febiger, 1991, pp. 17-23, incorporated by reference. The parameters calculated included “Cmax” (maximum serum concentration), “AUC” (area under the serum concentration time curve, extrapolated to infinite time, “CL” (total serum clearance) “Vss” (volume of distribution at steady state), “t½” (terminal half life), and “MRT” (mean residence time). Further analysis was carried out in accordance with Gibaldi, et al., Pharmacokinetics: New York, Marcel Dekker, 1982:45-111, incorporated by reference.

The mean half life of the antibody is about 65 hours, which is considerably less than the dosing interval between radioactive infusions. Hence, it was assumed that no radioactive material from week 1 remained in each patient at week 5, etc.

Pharmacokinetic parameters of ¹³¹I-BIBH 1 that were measured in patients who did not develop HAHA included terminal half life (65+28 hours); clearance (55+18 mL/hour), and Vss (4.3+1.0 L.). There was a trend toward a decrease of clearance, and increase of terminal half life of the antibody, with increasing doses of the material.

In 5 of the 16 patients for whom serum ¹³¹I-BIBH 1 profiles were available after more than one radioactive dose, changes in the form of serum ¹³¹I-BIBH 1 profiles occurred at weeks 5 and 9 as compared to week 1, indicating that there was altered disposition of BIBH 1. These were the same patients referred to supra, who developed a HAHA response. The changes coincided temporally, and in their extent with the development of HAHA. The appearance of HAHA caused decreases in clearance and increases in the volume of distribution, thus leaving the half-life essentially unaffected.

Five patients showed an increased in CL, with variations in Vss and t½ values.

Several conclusions can be drawn from the data secured in Examples 1-3, supra. First, the HAHA effect did not appear to impact the relatively short half life of the antibody, which is about 65 hours, or 2.7 days, and mean clearance of about 55 mL/hour. While there are few reports on the pharmacokinetics of humanized antibodies, half lives in the range of 8-25 days have been reported. It appears that the murine CDR and framework segments of BIBH 1 contribute significantly to the pharmacokinetics of this antibody.

The remaining parameters studied do appear to be similar to known data for physiological IgG. For example, compartmental analysis indicated 2-compartment kinetics of ¹³¹I-BIBH 1, with >95% of AUC under the terminal (beta) B elimination phase. The initial volume of distributors (V1), of 3.2L corresponds approximately to serum volume, while Vss was slightly higher (4.2L), indicating limited distribution of BIBH 1 outside the blood compartment. There was also a trend toward a decrease of clearance, and an increase of terminal half life of BIBH 1, with increasing doses, indicating non-linear kinetics. Normally, this is attributable to saturation of a clearance mechanism.

The short half life referred to supra is of special interest. There is limited exposure to bone marrow as a result of the short half life, rendering the antibody especially useful for radioimmunotherapy.

### EXAMPLE 5

Patients and Methods

Patients

Eligible patients had to fulfill the following criteria: (1) metastatic colorectal cancer UICC Stage IV (T1-4, N0-3, M1; or Dukes' D); progressive disease under at least two previous chemotherapy regimes and/or not eligible for conventional treatment; (2) progressive disease confirmed by CT or MRI; (3) ECOG performance status of ≦2; (4) life expectancy of ≧6 months; (5) age ≧18 years; (6) platelet count ≧100×10⁹/L, total leukocytes ≧2500/mm³, ALT/AST ≦4×upper limit of normal, total bilirubin ≦2.0 mg/dL, serum creatinine ≦2.0 mg/dL. Main exclusion criteria were: (1) active metastatic disease in the central nervous system; (2) exposure to an investigational agent, chemotherapy, immunotherapy or radiation therapy within 30 days prior to first sibrotuzumab infusion; (3) patients not fully recovered from surgery (incomplete healing); (4) previous administration of a murine, chimeric or humanized antibody and/or antibody fragment.

Study Design

This study was performed as an open-label, uncontrolled, multicenter trial. A two-stage design was adopted: the first 15 evaluable patients were to be accrued into the study with the aim to establish a response rate of BIBH 1 (stage 1). Should one partial (PR) or complete response (CR) or four patients with no change (NC) be noted among the first 15 patients eligible for analysis, additional 25 patients eligible for analysis would be entered (stage 2). Conversely, an early stopping rule would apply if no adequate responses (1 PR or 1 CR or 4 NC) were seen in the first 15 patients.

Patients received a fixed dose of 100 mg sibrotuzumab as an intravenous infusion once per week. A total of 12 administrations were scheduled. Depending on the response, a prolonged treatment was optionally possible. At moderate adverse reactions (CTC grade <3; adverse events were graded according to the Common Toxicity Criteria (CTC), version 2.0) the rate of infusion or the frequency of mAb therapy was to be reduced, grade 3 or 4 toxicity related to the test treatment would have led to the withdrawal of the patient from the trial.

Diagnostic Procedure

Tumor response was evaluated according to the WHO Disease Response Criteria at four weeks after the last administration of sibrotuzumab. Patients had to have at least 8 infusions of sibrotuzumab to be evaluable, unless early progression was noted. The size of tumor lesions was to be measured bi-dimensionally. Complete response meant the disappearance of all known disease and no evidence of new disease. For a partial response, the tumor had to be decreased by at least 50%. “No change” included stable disease, with an estimated increase of lesions of less than 25% and an estimated decrease of tumor size of less than 50%. Progressive disease was characterized by the appearance of any new lesions or estimated increase of 25% or more of existing lesions.

Safety was assessed through the incidence, nature and intensity of adverse events, the frequency of patient dropouts and on-study deaths. In addition, the development of HAHA was determined.

Pharmacokinetic and Immune Response Methods

Serum samples (2 mL) were taken from patients before each weekly infusion, 5 min after the end of infusions, on 3 further occasions between infusions distributed throughout the study, and at 2 and 4 weeks after the last infusion. Immunoreactive BIBH 1 was measured in all samples. HAHA were measured in the pre-infusion samples and after the last infusion. BIBH 1 was measured by a 2-site ELISA using anti-idiotype antibodies, and HAHAs were measured using an ELISA with sibrotuzumab bound to the solid phase and enzyme-labeled sibrotuzumab as a detection reagent. Pharmacokinetic data fitting was performed using a 2-compartment intravenous infusion model in the program NONMEM [13].

Results

A total of 25 patients with metastatic colorectal cancer were enrolled in this trial between February and October 2000. The baseline characteristics of the patients are listed in Table Table 1.

TABLE 1
Patient Baseline Characteristics

| | |
|---|---|
| Number | 25 |
| Sex (F/M) | 8/17 |
| Mean Age (years) | 62.7 |
| Median Age (years) | 63.0 |
| Age Range (years) | 52 to 76 |

| | Number of Patients |
|---|---|
| ECOG Performance | |
| 0 | 18 |
| 1 | 6 |
| 2 | 1 |
| Tumor Lesions | |
| Liver | 19 |
| Lung | 9 |
| Spleen | 1 |
| Pelvis | 2 |
| Lymph node | 3 |
| Prior Chemotherapy Treatment | |
| 5-FU/LV | 25 |
| Irinotecan | 14 |
| Oxaliplatin | 11 |
| Other | 14 |

Legend: 5-FU is 5-fluorouracil, and LV is leucovorin (folinic acid)

All enrolled patients were treated with the test drug; i.e. they received at least one infusion of sibrotuzumab (100 mg). Evaluable patients were to receive at least eight infusions of sibrotuzumab. Seventeen patients proved to be evaluable, thus exceeding the required number of patients for the interim analysis (at stage 1 of trial) by two.

Efficacy of Treatment

In total, 257 infusions were administered. Missing infusions could be attributed to patient discontinuation (34 infusions) and missed visits (9 infusions) In the post-study continued-use period, two patients received another 7 infusions. Evaluable patients (n=17) received 196 infusions. Two infusions were missing due to patient discontinuation and six infusions due to permitted transient interruptions of treatment.

The primary efficacy endpoint of this trial was the anti-tumor response four weeks following the last administration of sibrotuzumab. The assessment of tumor was to be based on measurable disease and on evaluable disease assessments. At baseline, patients had one or more measurable lesions, predominantly liver lesions (Table 1). The number and precise locations of the lesions were determined at the initial and final visits for each patient. For the 17 evaluable patients, at baseline a mean total lesion size was found to be 48.30 cm² (SD: 41.24). At the final visit, the mean value was increased by 50.07 cm², i.e. doubled.

Within the regular treatment cycle, progressive disease was noted in 15 out of 17 evaluable patients. The tumor status of two patients was evaluated as “no change” at this point of time. However, progressive disease was also observed in the continued-use period (post-study) in these patients. One of the patients showed evidence of tumor progression already after one additional infusion of sibrotuzumab, the second patient after six additional infusions.

On study, tumor measurements were planned and performed only at screening and in the post-treatment period. Therefore, time-to-progression, time-to loss of response and disease-free survival time could not be further evaluated.

Adverse Events

Weekly repeated intravenous dosing of 100 mg sibrotuzumab proved to be well tolerated and safe. Twenty-one patients experienced adverse events during the treatment period (including 28 days following the last infusion). Most of these events, including fatigue, anorexia and pain, are commonly encountered in cancer patients. Apart from thrombocytopenia (CTC grade 4) noted in one patient with a pre-existing thrombocytopenic condition, no hematological toxicities were observed. Eight patients experienced dyspnea, among them two cases classified as serious. One of the two patients experienced severe dyspnea (CTC grade 3) requiring hospitalization, but the event resolved on the same day. Worsening of tumor disease was diagnosed; however, no lung lesions were detected. Another patient developed severe dyspnea (CTC grade 3) due to the worsening of port-associated thrombosis requiring hospitalization and discontinuation of the test drug treatment. Five patients experienced adverse events, which were suspected to be causally related to the treatment. These adverse drug reactions were allergic reaction (bronchospasm)(one patient), rigor/chills (one patient), nausea (two patients), rigors, flush and dyspnea (one patient). A total of five patients dropped out from the study due to adverse events. One patient experienced severe septic cholangitis; two other patients had severe gastric bleeding and pain due to bleeding ulcer, respectively. Furthermore, two patients were withdrawn from the trial following to a drug-related hypersensitivity reaction and signs of dyspnea/flush, respectively, both of CTC grade 2. In both patients, antibodies against sibrotuzumab (HAHA) were detectable at the onset of the infusional events. HAHAs also developed in a third patient two weeks post-treatment, without any adverse clinical symptoms. Two patients died post-study due to aggravated malignant neoplasm and respiratory/renal insufficiency, respectively unrelated. Examination of laboratory data and of vital signs did not suggest any consistent, clinically significant changes associated with the administration of sibrotuzumab.

Pharmacokinetics

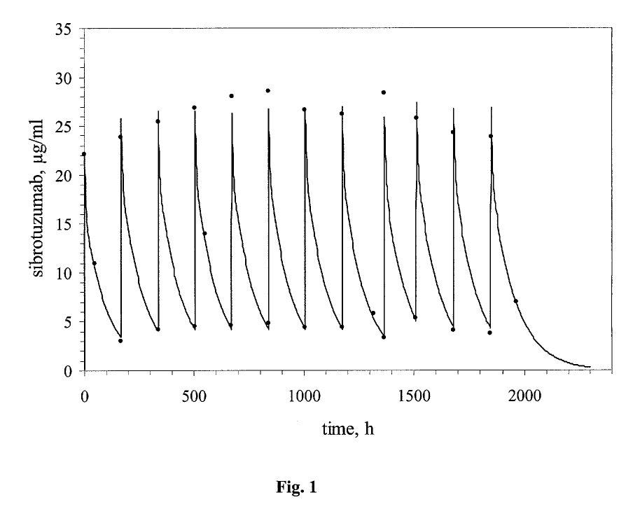

A typical 2-compartment pharmacokinetic data fit is shown in FIG. 1. Mean pharmacokinetic parameters of the 24 patients (±SD) were: clearance 39.8±19.8 mL/h, distribution half life 3.68±0.50 h, terminal half-life 127±55.4 h, initial volume of distribution 3.95±0.44 L, volume of distribution at steady state 5.82±0.66 L. Maximum and minimum BIBH 1 concentrations at steady state were 36.20±8.53 pg/mL and 11.4±7.7 μg/mL respectively. The area under the curve during a dosing interval at steady state was 3120±1440 μg·h/mL.

### REFERENCES

Boulianne G L, Hozumi N, and Shulman M J, Production of Functional Chimeric Mouse/Human Antibody, Nature 312: 643 (1984).

Chothia, C and Lesk, A M, Canonical Structures for the Hypervariable Regions of Immunoglobulins, J. Mol. Biol. 196: 901-917 (1987).

Hu S, Shively L, Raubitschek A, Sherman M, Williams L E, Wong J Y, Shively J E, and Wu A M, Minibody: A Novel Engineered Anti-Carcinoembryonic Antigen Antibody Fragment (single-chain Fv-CH3) which Exhibits Rapid, High-Level Targeting of Xenografts, Cancer Res 56(13), 3055-3061 (1996).

Huston J S, Levinson D, Mudgett-Hunter M, Tai M S, Novotny J, Margolies M N, Ridge R J, Bruccoleri R E, Haber E, Crea R, et al., Protein Engineering of Antibody Binding Sites: Recovery of Specific Activity in an Anti-Digoxin Single-Chain Fv Analogue Produced in Escherichia coli, Proc Natl Acad Sci USA 85(16): 5879-5883 (1988).

Kabat E A, Wu T T, Perry H M, Gottesman K S, and Foeller C, Sequences of Proteins of Immunological Interest (5th Ed.). NIH Publication No. 91-3242. U.S. Department of Health and Human Services, Public Health Service, National Institutes of Health, Bethesda, Md. (1991).

Kortt A A, Lah M, Oddie G W, Gruen C L, Bums J E, Pearce L A, Atwell J L, McCoy A J, Howlett G J, Metzger D W, Webster R G, and Hudson P J, Single-Chain Fv Fragments of Anti-Neuraminidase Antibody NC10 Containing Five- And Ten-Residue Linkers Form Dimers and with Zero-Residue Linker a Trimer, Protein Eng 10(4), 423-433 (1997).

LoBuglio A F, Wheeler R H, Trang J, Haynes A, Rogers K, Harvey E B, Sun L, Ghrayeb J, and Khazaeli M B, Mouse/Human Chimeric Monoclonal Antibody in Man: Kinetics and Immune Response, Proc. Natl. Acad. Sci. 86: 4220 (1989).

Lovejoy B, Choe S, Cascio D, McRorie D K, DeGrado W F, and Eisenberg D, Crystal Structure of a Synthetic Triple-Stranded Alpha-Helical Bundle, Science 259(5099), 1288-1293 (1993).

Pack P, Kujau M, Schroekh V, Knupfer U, Wenderoth R, Riesenberg R, Plückthun A, Improved Bivalent Miniantibodies, with Identical Avidity as Whole Antibodies, Produced by Whole-Density Fermentation of Escherichia coli, Biotechnology 11: 1271-1277 (1993).

Pack P, Müller K, Zahn R, and Plückthun A, Tetravalent Miniantibodies with High Avidity Assembling in Escherichia coli, JMol Biol 246(1), 28-34 (1995).

Perisic O, Webb P A, Holliger P, Winter G, Williams R L, Crystal Structure of a Diabody, a Bivalent Antibody Fragment, Structure 2: 1217-1226 (1994).

Remington's Pharmaceutical Sciences (1990), 18th ed. Mack Publ., Easton.

Riechmann L., Clark M., Waldmann H, and Winter G., Reshaping Human Antibodies for Therapy, Nature 332: 323 (1988).

We claim:

1. A method of treating a patient suffering from a pathological condition characterized by expression of FAPα, the method comprising administering to the patient a therapeutically effective amount of an antibody which specifically binds to FAPα.

2. The method of claim 1, wherein the antibody is a humanized antibody.

3. The method of claim 1, wherein the antibody comprises a variable region of the light chain selected from the group as set forth in SEQ ID NO: 2, SEQ ID NO: 4, or SEQ ID NO:6.

4. The method of claim 1, wherein the antibody has a variable region of the light chain encoded by a nucleotide sequence selected from the group as set forth in SEQ ID NO: 1, SEQ ID NO: 3, or SEQ ID NO: 5.

5. The method of claim 1, wherein the antibody comprises a variable region of the heavy chain as set forth in any one of SEQ ID NOs: 8, 10, 12, 14, or 16.

6. The method of claim 1, wherein the antibody has the variable region of the heavy chain encoded by a nucleotide sequence as set forth in any one of SEQ ID NOs: 7, 9, 11, 13, or 15.

7. The method of claim 1, wherein the antibody comprises a constant region of the light chain as set forth in SEQ ID NO: 18.

8. The method of claim 1, wherein the antibody comprises a constant region of the heavy chain as set forth in SEQ ID NO: 20.

9. The method of claim 1, wherein the antibody has the constant region of the light chain encoded by a nucleotide sequence as set forth in SEQ ID NO: 17.

10. The method of claim 1, wherein the antibody has the constant region of the heavy chain encoded by a nucleotide sequence as set forth in SEQ ID NO: 19.

11. The method of claim 1, wherein the antibody comprises the variable region of the light chain as set forth in SEQ ID NO: 2 and the variable region of the heavy chain as set forth in SEQ ID NO: 12.

12. The method of claim 1, wherein the antibody comprises the constant region of the light chain as set forth in SEQ ID NO: 20 and the constant region of the heavy chain as set forth in SEQ ID NO: 22.

13. The method of claim 1, wherein the antibody is antibody BIBH 1 or a fragment of the antibody which binds specifically to FAPα, wherein the antibody or antibody fragment is labeled with a therapeutically effective label.

14. The method of claim 1, wherein the pathological condition is cancer.

15. The method of claim 14, wherein the cancer is colorectal cancer, non-small cell lung carcinoma, breast cancer, or pancreatic cancer.

16. The method of claim 13, wherein the label is a radiolabel.

17. The method of claim 16, wherein the radiolabel is ¹³¹I, ⁹⁰Y, ¹⁸⁶Re, or 188Re.

18. The method of claim 17, wherein the radiolabel is ¹³¹I.

19. The method of claim 1, comprising administering the antibody or antibody fragment to the patient intravenously.

20. The method of claim 13, comprising administering the antibody or antibody fragment at a dose of from about 5 mg/m² to about 50 mg/m² body surface area.

21. The method of claim 20, comprising administering the antibody or antibody fragment at a dose of from about 10 mg/m² to about 40 mg/m² body surface area.

22. The method of claim 21, comprising administering the antibody or antibody fragment at a dose of from about 10 mg/m² to about 30 mg/m² body surface area.

23. The method of claim 22, comprising administering the antibody or antibody fragment at a dose of from about 20 mg/m² to about 30 mg/m² body surface area.

24. The method of claim 23, comprising administering the antibody or antibody fragment at a dose of about 25 mg/m² body surface area.

25. The method of claim 24, comprising administering the antibody or antibody fragment in combination with at least one additional therapeutic agent.

26. The method of claim 16, wherein the antibody has specific activity of from about 0.5 to about 15 mCi/mg.

27. The method of claim 26, wherein the antibody has specific activity of from about 0.5 to about 14 mCi/mg.

28. The method of claim 27, wherein the specific activity is from about 1 to about 10 mCi/mg.

29. The method of claim 28, wherein the specific activity is from about 1 to about 5 mCi/mg.

30. The method of claim 27, wherein the specific activity is from about 2 to about 6 mCi/mg.

31. The method of claim 28, wherein the specific activity is from 1 to 3 mCi/mg.

32. The method of claim 1, comprising administering the antibody in an aqueous solution at pH of from about 7 to about 8, and at a concentration of from about 0.5 to about 2.0 mg/mL.

33. A pharmaceutical composition comprising an antibody which specifically binds to FAPα, wherein the antibody is radiolabeled with ¹³¹I, ⁹⁰Y, ¹⁸⁶Re, or ¹⁸⁸Re, and wherein the antibody has specific activity of from about 0.5 to about 15 mCi/mg.

34. The pharmaceutical composition according to claim 33, wherein the specific activity is from about 0.5 to 14 mCi/mg.

35. The pharmaceutical composition according to claim 33, wherein the specific activity is from 1 to about 10 mCi/mg.

36. The pharmaceutical composition according to claim 33, wherein the specific activity is from 1 to 5 mCi/mg.

37. The pharmaceutical composition according to claim 33, wherein the specific activity is from 2 to 6 mCi/mg.

38. The pharmaceutical composition according to claim 33, wherein the specific activity is from 1 to 3 mCi/mg.

39. The pharmaceutical composition according to any one of claims 33 to 38, wherein the antibody is in an aqueous solution at pH of from about 7 to about 8, and at a concentration of from about 0.5 to about 2.0 mg/mL.

40. The pharmaceutical composition according to any one of claims 33 to 39, wherein the antibody is a humanized antibody.

41. The pharmaceutical composition according to any one of claims 33 to 40, wherein the antibody contains the variable region of the light chain selected from the group as set forth in SEQ ID NO: 2, SEQ ID NO: 4, or SEQ ID NO: 6.

42. The pharmaceutical composition according to any one of claims 33 to 41, wherein the antibody has the variable region of the light chain encoded by a nucleotide sequence selected from the group as set forth in SEQ ID NO: 1, SEQ ID NO: 3, or SEQ ID NO: 5.

43. The pharmaceutical composition according to any one of claims 33 to 42, wherein the antibody contains a variable region of the heavy chain as set forth in any one of SEQ ID NOs: 8, 10, 12, 14, or 16.

44. The pharmaceutical composition according to any one of claims 33 to 43, wherein the antibody has the variable region of the heavy chain is encoded by a nucleotide sequence as set forth in anyoneof SEQID NOs:7,9, 11, 13, or 15.

45. The pharmaceutical composition according to any one of claims 33 to 44, wherein the antibody contains a constant region of the light chain as set forth in SEQ ID NO: 18.

46. The pharmaceutical composition according to any one of claims 33 to 45, wherein the antibody contains a constant region of the heavy chain as set forth in SEQ ID NO: 20.

47. The pharmaceutical composition according to any one of claims 33 to 46, wherein the antibody has the constant region of the light chain is encoded by a nucleotide sequence as set forth in SEQ ID NO: 17.

48. The pharmaceutical composition according to any one of claims 33 to 47, wherein the antibody has the constant region of the heavy chain is encoded by a nucleotide sequence as set forth in SEQ ID NO: 19.

49. The pharmaceutical composition according to any one of claims 33 to 48, wherein the antibody contains the variable region of the light chain as set forth in SEQ ID NO: 2 and the variable region of the heavy chain as set forth in SEQ ID NO: 12.

50. The pharmaceutical composition according to any one of claims 33 to 49, wherein the antibody additionally contains the constant region of the light chain as set forth in SEQ ID NO: 20 and the constant region of the heavy chain as set forth in SEQ ID NO: 22.

51. The pharmaceutical composition according to any one of claims 33 to 50, wherein the radiolabel is ¹³¹I.
